(19) United States
(12) Patent Application Publication
(10) Pub. No.: US 2020/0171188 A1
(43) Pub. Date: Jun. 4, 2020

(54) Safety Cabinet and Method for Sterilizing Same

(71) Applicant: Hitachi Industrial Equipment Systems Co., Ltd., Chiyoda-ku, Tokyo (JP)

(72) Inventors: Takeshi KANEKO, Tokyo (JP); Hirotoshi SATO, Tokyo (JP)

(21) Appl. No.: 16/615,153

(22) PCT Filed: Jul. 14, 2017
(86) PCT No.: PCT/JP2017/025770

§ 371 Date: Nov. 20, 2019

(51) Int. Cl. A61L 2/20; A61L 2/24; B01L 1/00; C12M 1/00

(52) U.S. Cl. CPC A61L 2/208; A61L 2/24; B01L 1/50; A61L 2202/14; A61L 2202/122; A61L 2202/121; A61L 2202/15; C12M 47/16

(57) ABSTRACT

There is provided a safety cabinet in which a good operability is attained and it is possible to secure a sterility assurance level. There is provided a safety cabinet including an operation chamber, a front door that covers part of an opening of a front surface of the operation chamber, an operation opening below the front door, into which an operator can insert an hand and perform an operation, and a front slit, which takes in air inside the operation chamber and air in a room through the operation opening, on a front surface side of a lower portion of the operation chamber, the cabinet including sterilization gas generating means; and sealing means for sealing the operation opening during a sterilization operation.

### TECHNICAL FIELD

The present invention relates to a safety cabinet used for the preparation of cells or the like, and a method for sterilizing the same.

### BACKGROUND ART

When cells or microorganisms are handled in a research of pathogens or the like or in regenerative medicine or the like, an isolator or a safety cabinet is used.

In the isolator which is a closed system, an operator can perform an operation via an operation glove from outside an operation chamber which is isolated. In the isolator, when a handled patient tissue is changed, or the type of a handled pathogen is changed, it is necessary to sterilize the operation chamber or the glove by cleaning and disinfecting the inside of the operation chamber or the glove used in the operation. The sterilization is performed by supplying a sterilization gas to the operation chamber or the glove.

In the safety cabinet (class II cabinet for the countermeasure of biohazard) which is an open system, purified air from which dust, pathogens, and the like are filtered by a HEPA filter or the like is supplied to an operation chamber from an upper outlet port of the operation chamber formed inside the apparatus. Then, together with air in the operation chamber, air in a room where the safety cabinet is disposed is taken in from an operation bed front intake port, which is formed on a front side of an operation bed that is a lower surface of the operation chamber, through an operation opening formed in a front surface of the operation chamber, and inlet airflows are formed in the operation opening. In addition, when the intake air is exhausted outside the safety cabinet, air containing pathogens and the like is filtered by an exhaust HEPA filter or the like. The HEPA filter is the abbreviation of a high efficiency particulate air filter. A pathogen or the like, which is handled inside the operation chamber, is prevented from leaking outside the safety cabinet which is caused by the inlet airflows formed in the operation opening to infect an operator and to spread to the environment.

Patent Document 1 illustrates one example of the isolator, and Patent Document 2 illustrates one example of the safety cabinet.

### CITATION LIST

### Patent Document

Patent Document 1: JP 2010-69255 A

Patent Document 2: JP 2009-119391 A

### SUMMARY OF THE INVENTION

### Problems to be Solved by the Invention

In the isolator which is a closed system, it is possible to secure a sterility assurance level by sterilizing the operation chamber or the operation glove by supplying the sterilization gas thereto; however, since it is necessary to perform an operation via the operation glove attached to a front door, there is a problem in securing operability.

On the contrary, in the safety cabinet, since the operator inserts the hands from the operation opening below a front door, and performs an operation, a good operability is attained; however, in order to sterilize the operation chamber, it is necessary to sterilize the entirety of the room where the safety cabinet is installed, or in regular inspection, it is necessary for a service engineer to perform sterilization in a state where the front surface of the operation chamber is covered, and thus there is a problem in sterilizing the apparatus and securing the sterility assurance level.

Herein, the sterility assurance level (SAL) refers to the maximum survival probability of contaminating bacteria which is estimated to exist in a sterilized product processed in a proper sterilization step, and is expressed by 10⁻ⁿ. Currently, SAL: 10⁻⁶ is adopted internationally, and implies that the probability where microorganisms survive in a sterilized object after a sterilization operation is one millionth.

An object of the present invention is to provide a safety cabinet in which a good operability is attained and it is possible to secure a sterility assurance level.

### Solutions to Problems

In order to solve the problems, as one example of a “safety cabinet” of the present invention, there is provided a safety cabinet including an operation chamber, a front door that covers part of an opening of a front surface of the operation chamber, an operation opening below the front door, into which an operator can insert an hand and perform an operation, and a front slit, which takes in air inside the operation chamber and air in a room through the operation opening, on a front surface side of a lower portion of the operation chamber, the cabinet including sterilization gas generating means; and sealing means for sealing the operation opening during a sterilization operation.

In addition, as one example of a method for sterilizing a safety cabinet of the present invention, there is provided a method for sterilizing a safety cabinet including an operation chamber, a front door that covers part of an opening of a front surface of the operation chamber, an operation opening below the front door, into which an operator can insert an hand and perform an operation, and a front slit, which takes in air inside the operation chamber and air in a room through the operation opening, on a front surface side of a lower portion of the operation chamber, the method including a step of sealing the operation opening; a step of performing an airtightness test on the safety cabinet including the operation chamber; and a step of performing sterilization by supplying a sterilization gas to a flow path including the operation chamber when airtightness is confirmed by the airtightness test.

### Effects of the Invention

According to the present invention, it is possible to provide the safety cabinet in which a good operability is attained and it is possible to secure the sterility assurance level.

Tasks, configurations, and effects other than those described above become apparent from the description of the following embodiments.

### BRIEF DESCRIPTION OF THE DRAWINGS

FIG. 1 is a front view illustrating one example of a safety cabinet of Example 1.

FIG. 2 is a left central cross-sectional view of the safety cabinet in FIG. 1.

FIG. 3 is a plan view of an upper portion of the safety cabinet in FIG. 1.

FIG. 4 is an enlarged view of the vicinity of an opening of a front surface of the safety cabinet in FIG. 1.

FIG. 5 is a configuration block diagram related to a sterilization operation of the safety cabinet of Example 1.

FIG. 6 is a flowchart illustrating the sterilization operation of the safety cabinet of Example 1.

FIG. 7 is an enlarged view of the vicinity of an opening of the front surface in a modification example of the safety cabinet in FIG. 1.

FIG. 8 is a plan view illustrating one example of a safety cabinet of Example 2.

FIG. 9 is a left central cross-sectional view of the safety cabinet of Example 2.

FIG. 10 is a plan view of an upper portion of the safety cabinet of Example 2.

FIG. 11 is a front view of a safety cabinet of Example 3.

FIG. 12 is a plan view of an upper portion of the safety cabinet of Example 3.

FIG. 13 is a plan view of an upper portion in a modification example of the safety cabinet of Example 3.

### MODE FOR CARRYING OUT THE INVENTION

Embodiments of the present invention will be described with reference to the drawings. Incidentally, in each drawing for describing the embodiments, as far as possible, the same names and reference signs will be assigned to the same configuration elements, and the repeated descriptions thereof will be omitted.

### Example 1

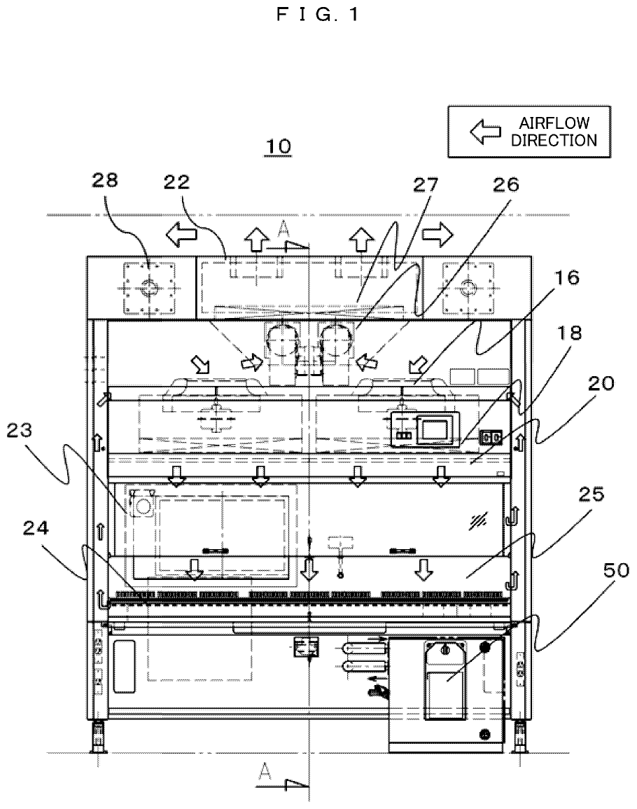

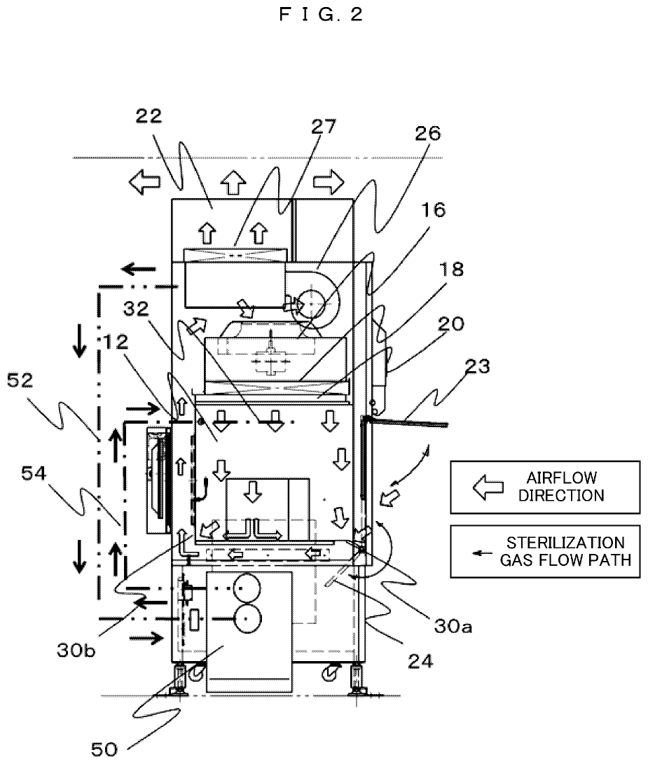

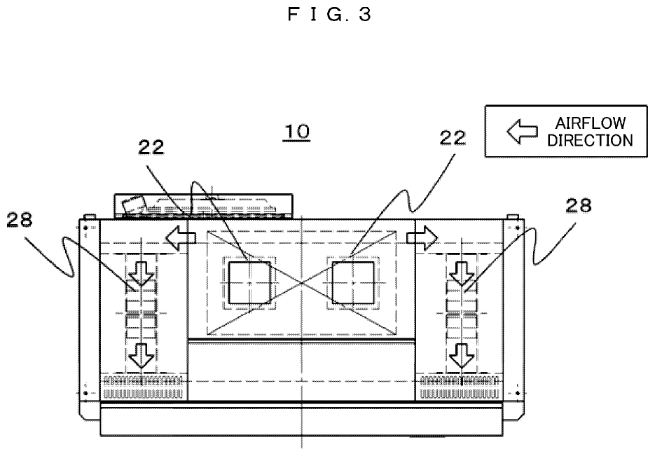

FIG. 1 a front view illustrating one example of a safety cabinet of Example 1, FIG. 2 is a left central cross-sectional view thereof (view taken along line A-A in FIG. 1), and FIG. 3 is a plan view seen from above.

An operation chamber 12 in which a specimen such as a pathogen is handled is provided inside a case (housing). A front door 23 which covers part of an opening is provided in a front surface of the operation chamber 12. An operation opening 25, into which an operator can insert the hands and perform an operation, is provided below the front door 23. A circulation fan 16 is provided in an upper portion of the operation chamber 12, a circulation HEPA filter 18 and a punching plate 20 are provided downstream of the circulation fan 16, and a rectified flow of purified air is supplied to the operation chamber 12. A front slit (front intake port) 30a is provided on a front surface side of a lower portion of the operation chamber 12, and takes in air inside the operation chamber and air in a room through the operation opening 25. In addition, air inside the operation chamber is taken in from a back slit (rear intake port) 30b provided on a back surface side. The air taken in through the slits 30a and 30b returns upstream of the circulation fan 16 through ducts provided on a side surface and a back surface of the operation chamber, so that the air circulates. Part of the air is delivered to an exhaust HEPA filter 27 by an exhaust fan 26, and is exhausted to the outside from an airtight damper 22 for exhaust which is provided on an upper surface of the case. The front door 23 of the operation chamber 12 is made of, for example, glass or resin. The operator can see an operation, which is performed with the hands, therethrough. In addition, as illustrated by an arrow in FIG. 2, the front door 23 is configured so as to be able to rotate to be open and closed. During an operation, the front door 23 is closed to secure an air barrier, and when equipment is to be put in, the front door 23 is open, and the equipment or the like can be taken in and out. In the safety cabinet of FIG. 1, two circulation fans 16, two circulation HEPA filters 18, two exhaust fans 26, two exhaust HEPA filters 27, two airtight dampers 22 for exhaust, and the like are provided bisymmetrically; however, only one set of each may be provided.

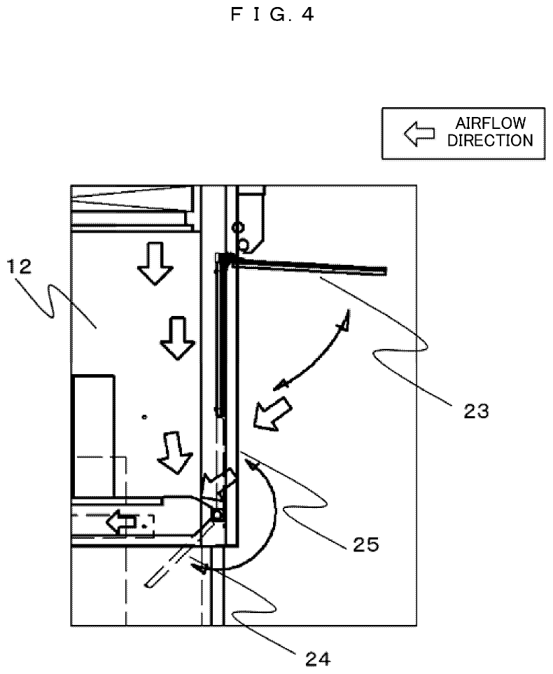

A sterilization airtight cover 24 which closes the opening 25 of the front surface of the operation chamber is provided as a distinguishing configuration of the present example. As illustrated in FIG. 4 which is an enlarged view of the vicinity of the opening of the front surface, the sterilization airtight cover 24 which rotates as illustrated by an arrow is provided below the opening of the front surface of the operation chamber. Then, it is possible to seal the opening of the front surface of the operation chamber by rotating and closing the sterilization airtight cover 24 and the front door 23.

Then, the safety cabinet of the present example includes a sterilization gas generation apparatus 50 which is to be used for sterilization. A sterilization gas such as hydrogen peroxide gas is supplied from a condition (outgoing) path 54 to the operation chamber 12, and is sprayed from a sterilization gas inlet port 32. The sterilization gas circulates through a flow path of the safety cabinet, and part of the sterilization gas returns to the sterilization gas generation apparatus 50 through a condition (returning) path 52. A safety cabinet 10 is provided with an aeration path for removing the sterilization gas after a sterilization step ends. For example, as illustrated in FIG. 3 which is a plan view, a catalyst unit 28 which adsorbs the sterilization gas is provided as the aeration path, and adsorbs the sterilization gas when airflows from the exhaust fan 26 pass therethrough.

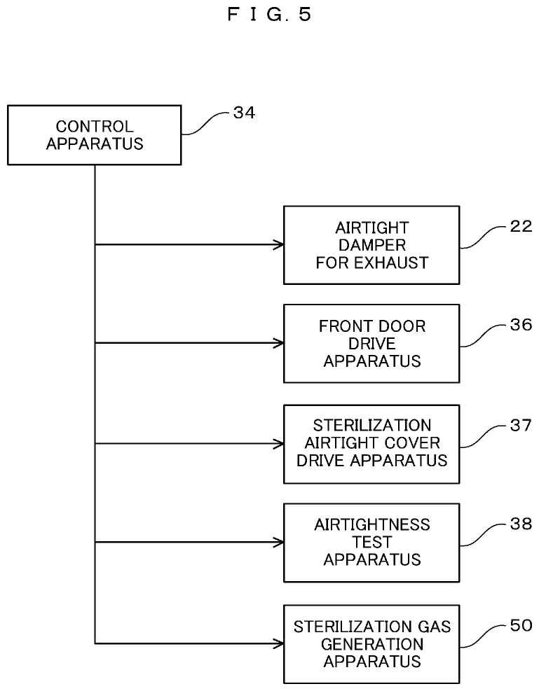

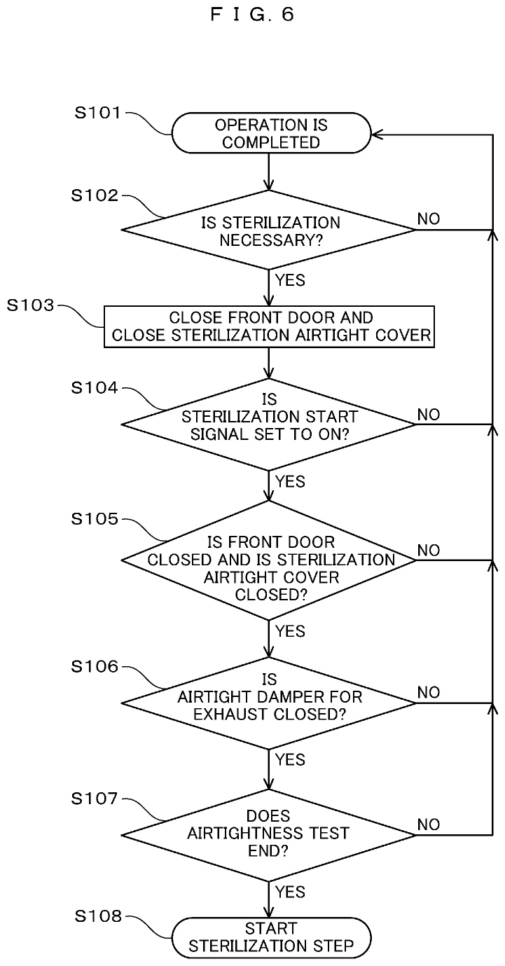

FIG. 5 illustrates a configuration block diagram related to a sterilization operation of the present example. A control apparatus 34 of the safety cabinet sends a control signal for closing the airtight damper 22 for exhaust in the sterilization operation. In addition, the control apparatus 34 sends a control signal for closing the front door to a front door drive apparatus 36 that opens and closes the front door 23, and sends a control signal for closing the sterilization airtight cover to a sterilization airtight cover drive apparatus 37 that opens and closes the sterilization airtight cover 24. In addition, the control apparatus 34 sends a control signal for operating an airtightness test apparatus 38. The airtightness test apparatus 38 is formed of, for example, a pressurizing apparatus that increases the atmospheric pressure of the operation chamber or the like, and atmospheric pressure detection means for detecting the atmospheric pressure of the operation chamber or the like. The airtightness test apparatus 38 tests an airtight state of the operation chamber or the like by operating the pressurizing apparatus to increase the atmospheric pressure of the operation chamber or the like, and detecting, by the atmospheric pressure detection means, whether or not the atmospheric pressure thereafter has decreased. For example, the pressurizing apparatus is configured such that an air cylinder is connected to the safety cabinet via a pipe and an electromagnetic valve is provided in part of the pipe. The pressurizing apparatus delivers air in the air cylinder to the safety cabinet and pressurizes the air by opening the electromagnetic valve. Incidentally, the airtightness test apparatus 38 is not limited to the configuration, and may be any type of device as long as the device can test whether or not the operation chamber or the like is in an airtight state. Furthermore, the control apparatus 34 sends a control signal to the sterilization gas generation apparatus 50 to generate a sterilization gas such as hydrogen peroxide gas and to supply the sterilization gas to the operation chamber or the like. Incidentally, part of an operation of the apparatuses may be performed manually. An operation flow of the apparatuses will be described later in the description of FIG. 6.

In FIGS. 1 and 2, outlined arrows indicate an airflow direction (flow of air) in a normal operation where a specimen is processed inside the operation chamber. Air is delivered to a pressurizing chamber by the circulation fan 16, and a rectified flow of purified air is delivered into the operation chamber 12 by the circulation HEPA filter 18 and the punching plate 20 downstream of the circulation fan 16. Air in a room where the safety cabinet is disposed is taken in from the front slit 30a, which is formed on a front side of a lower surface of the operation chamber, through the operation opening 25 formed in the front surface of the operation chamber, air in the operation chamber 12 is taken in together, and inlet airflows are formed in the operation opening 25. The air in the operation chamber 12 is taken in also from the back slit 30b of a lower portion of the back surface of the operation chamber. The intake air returns upstream of the circulation fan 16 through the ducts provided on the side surface and the back surface of the operation chamber, so that the intake air circulates through the flow path of the safety cabinet. Part of the air is exhausted from the airtight damper 22 for exhaust to the outside through the exhaust HEPA filter 27 by the exhaust fan 26. As described above, it is possible to prevent the contamination of the specimen under operation by supplying a rectified flow of the purified air to the operation chamber 12.

When a handled patient tissue is changed, or the type of a handled pathogen or the like is changed, it is necessary to sterilize the inside of the operation chamber. The airtight damper 22 for exhaust is closed during sterilization. In addition, in addition to closing the front door 23, the sterilization airtight cover 24 is closed to shut off the flow of air between the safety cabinet and the outside. For example, hydrogen peroxide gas generated by the sterilization gas generation apparatus 50 is supplied from the condition (outgoing) path 54 to the operation chamber 12 of the safety cabinet. Then, the sterilization gas circulates from the operation chamber to the circulation fan 16 and the circulation HEPA filter 18 through the ducts, and part of the sterilization gas returns to the sterilization gas generation apparatus 50 through the condition (returning) path 52. As described above, the sterilization gas circulates through the safety cabinet including the operation chamber, and thus it is possible to sterilize the safety cabinet including the operation chamber.

Incidentally, in FIG. 2, the sterilization gas is directly supplied to the operation chamber 12, but may be supplied to a flow path through which air circulates or, for example, may be supplied to an intake port of the circulation fan 16.

In addition, in the present example, a HEPA filter has been described as a filter; however, any type of air filter may be used as long as purified air from which dust, pathogens, and the like are filtered can be supplied through the air filter.

The sterilization step is performed as follows.

(1) Dehumidification Step

Humidity is lowered by dry air. The required concentration of a sterilization gas (for example, hydrogen peroxide gas) is kept equal to or less than a saturation level in the following conditioning step and the following decontamination step by lowering the humidity. Returning air is dried and heated via a dry cartridge.

(2) Conditioning Step

While a sterilizing agent is injected into the airflows, the dry air continues to circulate until immediately before the sterilization gas leaves an instrument. The conditioning step is a step for rapidly reaching a target sterilization concentration.

(3) Decontamination Step

For a specific time, the entire concentration of the sterilization gas inside the safety cabinet is maintained by the sterilizing agent, and the operation chamber, the HEPA filter, or the like is sterilized.

(4) Aeration Step

The injection of the sterilizing agent is stopped, and the aeration path including the catalyst unit which adsorbs the sterilization gas is connected. Then, the dry air circulates for a predetermined time, and the concentration of the sterilization gas inside the safety cabinet and a connection hose is lowered.

FIG. 6 illustrates a flow from the completion of an operation such as the preparation of cells to the start of the sterilization step. In Step S101, an operation is completed. Incidentally, during the operation, the front door 23 is closed, and the sterilization airtight cover 24 is open. In step S102, it is determined whether or not sterilization is necessary, and if it is determined that sterilization is necessary, as illustrated in Step S103, it is necessary to close the sterilization airtight cover 24 in a state where the front door 23 is closed. For this reason, in Step S104, an operator sets a sterilization start signal to ON. In Step S105, the front door 23 is closed, and the sterilization airtight cover 24 is closed. Then, in Step S106, the airtight damper (electric damper) 22 for exhaust is closed. Then, in Step S107, an airtightness test is performed on the safety cabinet including the operation chamber. In the airtightness test, it is inspected whether or not the airtightness is kept by increasing the pressure of the operation chamber or the like and detecting the leak of air. If the airtightness test ends, the sterilization step starts in Step S108. Incidentally, if the sterilization step is completed, a sterilization completion signal is sent, and the end of the sterilization step is displayed by a buzzer, a lamp, a touch panel, or the like, but not illustrated in the figures.

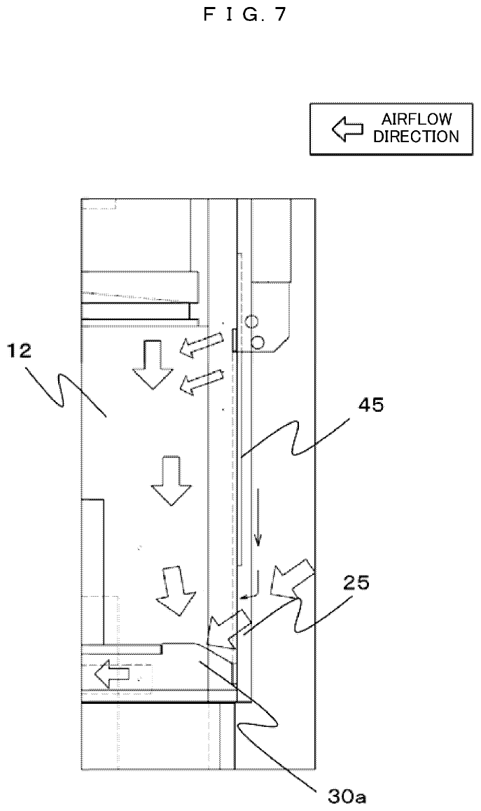

FIG. 7 illustrates an enlarged view of the vicinity of an opening of a front surface in a modification example of the safety cabinet of the present example. The modification example is an example where the configuration of the front door 23 and the sterilization airtight cover 24 in FIG. 4 is modified. In the modification example, a slide type front door 45 is provided as a front door provided in the front surface of the operation chamber 12. The slide type front door 45 slides in a vertical direction. FIG. 7 illustrates a state during an operation, in which the slide type front door 45 has the opening 25 of the front surface therebelow and covers above the opening of the operation chamber 12. During sterilization, the slide type front door 45 slides to a lower end, and seals the entirety of the opening of the front surface of the operation chamber. Incidentally, if necessary, a packing may be provided around the opening, or pressing means for pressing the slide type front door in the direction of the packing may be provided for the sterilization gas not to leak.

According to the present example, the safety cabinet including the operation opening in a lower portion of the front surface is configured such that sealing means for closing the operation opening during sterilization is provided and the sterilization gas can be supplied, and thus it is possible to provide the safety cabinet in which an operator can perform sterilization after an operation, a good operability is attained, and it is possible to secure a sterility assurance level. In addition, it is possible to save labor by automating the sterilization operation of the safety cabinet.

### Example 2

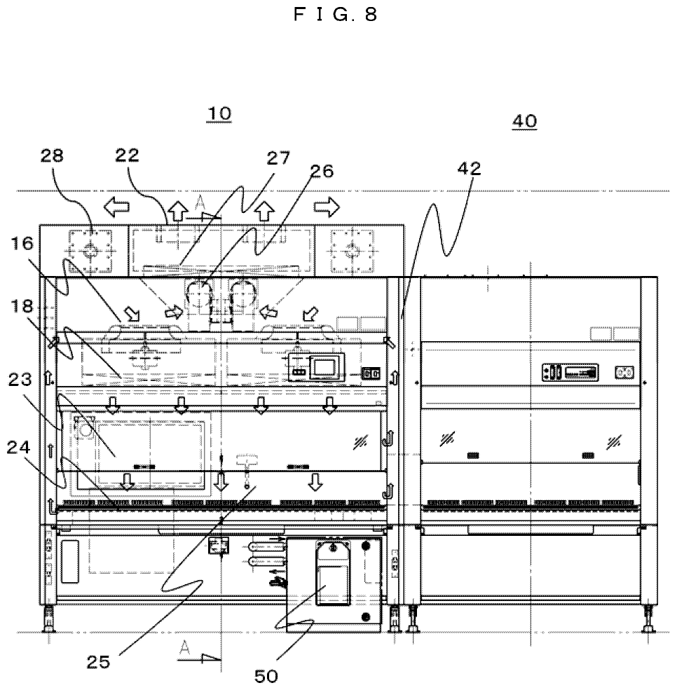

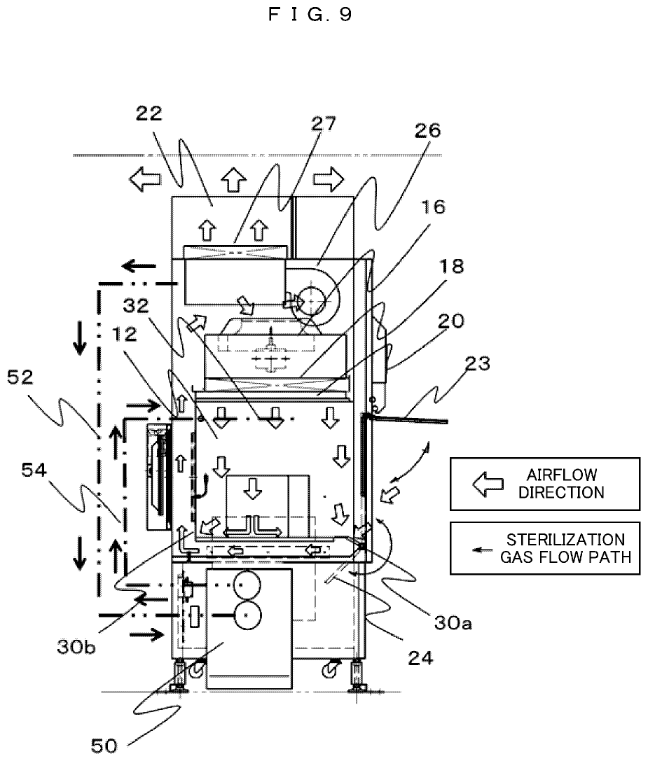

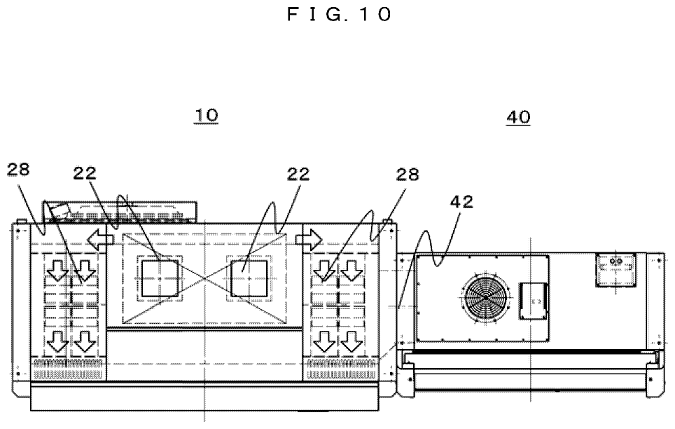

FIG. 8 is a front view illustrating one example of a safety cabinet of Example 2, FIG. 9 is a left central cross-sectional view thereof (view taken along A-A arrows in FIG. 8), and FIG. 10 is a plan view seen from above. In Example 2, the safety cabinet 10 for manufacture (preparation) which is used for the preparation of cells or the like and two safety cabinets 40 for inspection are coupled to each other.

The safety cabinet 10 for manufacture (preparation) requires that an operation chamber is a purified space, has a high airtightness, and can be sterilized. On the contrary, the safety cabinet 40 for inspection requires that an operation chamber is a purified space and a specimen such as cells is contained inside the operation chamber. Therefore, the safety cabinet 10 for manufacture (preparation) and the safety cabinet 40 for inspection are coupled to each other via a coupling portion 42, and an opening and closing door is provided in the coupling portion. The opening and closing door is normally closed, and is to be open when the specimen is moved between the safety cabinet 10 for manufacture (preparation) and the safety cabinet 40 for inspection in an inspection step. In addition, during sterilization, the opening and closing door is sealed. During an operation and sterilization, the operation of the safety cabinet 10 for manufacture (preparation) is the same as that in Example 1.

According to the present example, the safety cabinet for manufacture (preparation) and the safety cabinet for inspection are coupled to each other, and different steps for manufacture (preparation) and inspection are shared via the coupling portion in a vacuum state, and thus it is possible to transfer the specimen in the purified spaces without contaminating the specimen, and it is possible to limit the application of a sterilization space only to the safety cabinet for manufacture (preparation). Therefore, it is possible to reduce a sterilization time.

### Example 3

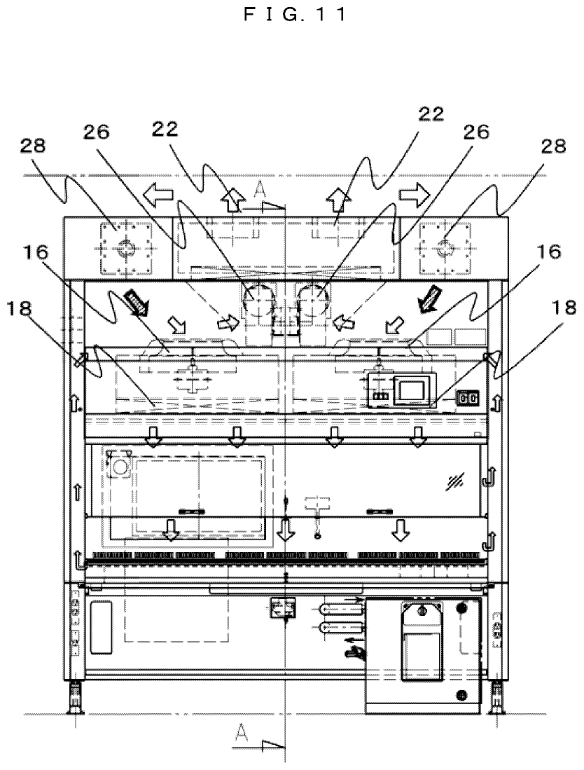

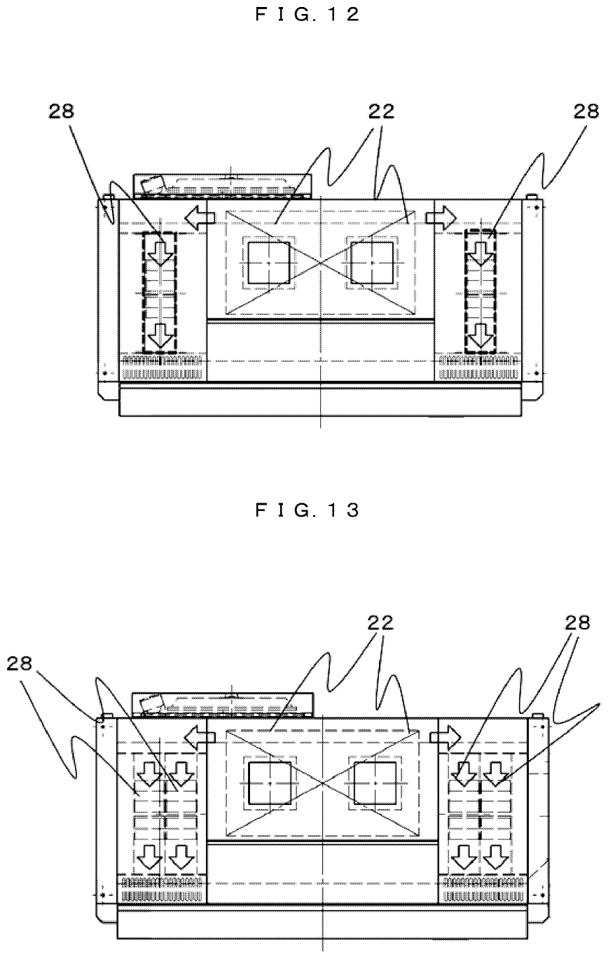

FIG. 11 is a front view of a safety cabinet of Example 3 of the present invention, and FIG. 12 is a plan view of an upper portion. The present example is intended to be able to reduce a sterilization time, particularly the time of the aeration step.

The present example is configured such that the H₂O₂ catalyst unit 28 is disposed in a flow path for airflows induced by the exhaust fan 26 when the airtight damper 22 for exhaust is closed, and airflows which have passed through the H₂O₂ catalyst unit 28 are drawn into the intake port of the circulation fan 16.

The airtight damper 22 for exhaust is fully closed during sterilization. In the aeration step during sterilization, as illustrated by arrows in FIG. 12, airflows from the exhaust fan 26 flow to the H₂O₂ catalyst units 28 on both sides of the airtight damper 22 for exhaust, and airflows which have passed through the H₂O₂ catalyst units 28 are drawn into the intake ports of the circulation fans 16. By using a catalyst that lowers the concentration of H₂O₂ gas (hydrogen peroxide gas) which is a sterilization gas to 1 ppm or less in one pass as the H₂O₂ catalyst unit 28, air which does not almost contain the H₂O₂ gas having a concentration of 1 ppm or less is drawn to the circulation fan 16, and passes through a filter material of the circulation HEPA filter 18 which has adsorbed the remaining gas. For this reason, it is possible to efficiently lower the concentration of the H₂O₂ sterilization gas, and to reduce the sterilization time of the aeration step. In addition, it is possible to also prevent the H₂O₂ sterilization gas from remaining in the filter material of the circulation HEPA filter 18. In addition, since the exhaust fan 26 can be used as a fan for circulating the H₂O₂ gas, and there can be a path through the exhaust HEPA filter, it is also possible to eliminate the H₂O₂ gas remaining in the exhaust HEPA filter. Furthermore, if the airtight damper 22 for exhaust is open and the exhaust fan 26 is in operation, part of the airflows flows to the H₂O₂ catalyst unit 28, and it is possible to lower the concentration of the H₂O₂ gas.

FIG. 13 illustrates a modification example of Example 3. In the modification example, the number of the H₂O₂ catalyst units 28 is increased, and the H₂O₂ catalyst units 28 are disposed side by side. It is possible to further reduce an aeration time by increasing the number of revolutions of the exhaust fan 26 and increasing a flow rate during aeration.

According to the present example, the catalyst unit is disposed in the flow path for airflows induced by the exhaust fan when the airtight damper for exhaust is closed, and airflows which have passed through the catalyst unit are drawn into the intake port of the circulation fan through the flow path, and thus it is possible to efficiently lower the concentration of the sterilization gas, and to reduce the sterilization time of the aeration step. In addition, it is possible to also prevent the sterilization gas from remaining in the filter material of the circulation HEPA filter.

In addition, if during aeration, the flow rate of the exhaust fan is increased and the circulation fan operates intermittently or operates at a low flow rate, since the circulation efficiency of a circulation path of the catalyst unit improves, it is possible to reduce the time of the aeration step.

Furthermore, since the catalyst unit is accommodated inside the safety cabinet, it is possible to make the appearance look cleaner compared to a case where the catalyst unit is provided outside. In addition, it is not necessary to perform on-site work such as connecting the catalyst unit to the safety cabinet after the safety cabinet is installed.

### REFERENCE SIGNS LIST

10 Safety cabinet (for manufacture)
12 Operation chamber
16 Circulation fan
18 Circulation HEPA filter
20 Punching plate
22 Airtight damper for exhaust
23 Front door
24 Sterilization airtight cover
25 Operation opening
26 Exhaust fan
27 Exhaust HEPA filter
28 H₂O₂ catalyst unit
30a Front slit
30b Back slit
32 Sterilization gas inlet port
34 Control apparatus
36 Front door drive apparatus
37 Sterilization airtight cover drive apparatus
38 Airtightness test apparatus
40 Safety cabinet (for inspection)
42 Coupling portion
45 Slide type front door
50 Sterilization gas generation apparatus
52 Condition (returning) path
54 Condition (outgoing) path

1. A safety cabinet including an operation chamber, a front door that covers part of an opening of a front surface of the operation chamber, an operation opening below the front door, into which an operator can insert an hand and perform an operation, and a front slit, which takes in air inside the operation chamber and air in a room through the operation opening, on a front surface side of a lower portion of the operation chamber, the cabinet comprising:
sterilization gas generating means; and
sealing means for sealing the operation opening during a sterilization operation.

2. The safety cabinet according to claim 1,
wherein the sealing means for sealing the operation opening includes
the front door that rotates to cover part of the opening of the front surface of the operation chamber, and
a sterilization airtight cover that rotates to cover a remainder of the opening of the front surface of the operation chamber.

3. The safety cabinet according to claim 1,
wherein the sealing means for sealing the operation opening includes a slide type front door that slides downward to cover an entire surface of the opening of the front surface of the operation chamber.

4. The safety cabinet according to claim 1, further comprising:
an airtight damper for exhaust which is sealed during the sterilization operation.

5. The safety cabinet according to claim 1, further comprising:
a circulation fan;
an air filter; and
a punching plate,
wherein the safety cabinet supplies a rectified flow of purified air to the operation chamber.

6. The safety cabinet according to claim 1,
wherein the sterilization gas generating means generates a hydrogen peroxide gas as a sterilization gas.

7. The safety cabinet according to claim 1, further comprising:
a control apparatus that controls the sterilization operation,
wherein during the sterilization operation, the control apparatus
supplies a control signal for closing the front door to a front door drive apparatus that opens and closes the front door,
supplies a control signal for closing a sterilization airtight cover to a sterilization airtight cover drive apparatus that opens and closes the sterilization airtight cover covering a remainder of the opening of the front surface of the operation chamber,
supplies a control signal for performing an airtightness test to an airtightness test apparatus that performs the airtightness test on the safety cabinet including the operation chamber, and
supplies a control signal for operating the sterilization gas generation apparatus to supply a sterilization gas to a flow path including the operation chamber after airtightness is confirmed.

8. The safety cabinet according to claim 7,
wherein during the sterilization operation, the control apparatus further supplies a control signal for closing an airtight damper for exhaust.

9. The safety cabinet according to claim 1,
wherein a safety cabinet for inspection is further coupled to the safety cabinet via a coupling portion including an opening and closing door.

10. The safety cabinet according to claim 4, further comprising:
an exhaust fan;
a catalyst unit that is disposed in a flow path for an airflow induced by the exhaust fan when the airtight damper for exhaust is closed; and
a flow path through which the airflow which has passed through the catalyst unit is drawn into an intake port of a circulation fan supplying air to the operation chamber.

11. The safety cabinet according to claim 10,
wherein a plurality of the catalyst units are disposed side by side in the flow path for the airflow induced by the exhaust fan.

12. A method for sterilizing a safety cabinet including an operation chamber, a front door that covers part of an opening of a front surface of the operation chamber, an operation opening below the front door, into which an operator can insert an hand and perform an operation, and a front slit, which takes in air inside the operation chamber and air in a room through the operation opening, on a front surface side of a lower portion of the operation chamber, the method comprising:
a step of sealing the operation opening;
a step of performing an airtightness test on the safety cabinet including the operation chamber; and
a step of performing sterilization by supplying a sterilization gas to a flow path including the operation chamber when airtightness is confirmed by the airtightness test.

13. The method for sterilizing a safety cabinet according to claim 12, further comprising:
a step of closing an airtight damper for exhaust.

14. The method for sterilizing a safety cabinet according to claim 12,
wherein in the step of sealing the operation opening,
the front door rotates to cover part of the opening of the front surface of the operation chamber, and
a sterilization airtight cover rotates to cover a remainder of the opening of the front surface of the operation chamber.

15. The method for sterilizing a safety cabinet according to claim 12,
wherein in the step of sealing the operation opening, a slide type front door slides downward to cover an entire surface of the opening of the front surface of the operation chamber.
